(19) United States
(12) Patent Application Publication
(10) Pub. No.: US 2015/0208980 A1
(43) Pub. Date: Jul. 30, 2015

(54) ALIGNMENT FRAME FOR WEIGHT-BEARING IMAGING OF KNEE

(71) Applicant: CurveBeam LLC, Warrington, PA (US)

(72) Inventors: Neil Segal, Overland Park, KS (US); Frank Speranza, Warminster, PA (US)

(73) Assignee: CURVEBEAM LLC, Warrington, PA (US)

(21) Appl. No.: 14/604,231

(22) Filed: Jan. 23, 2015

Related U.S. Application Data
(60) Provisional application No. 61/932,535, filed on Jan. 28, 2014.

(51) Int. Cl. A61B 5/00; A61B 6/04; A61B 6/03; A61B 5/055

(52) U.S. Cl. CPC A61B 5/4585; A61B 5/702; A61B 5/0555; A61B 6/04; A61B 6/032

(57) ABSTRACT

An alignment frame for use in weight bearing imaging of a knee or knees, the alignment frame includes a base with a support extending upward from a base. A thigh positioner is attached to the support. The thigh positioner includes a panel portion for contact by a user's thigh, and an imaging portion located below the panel portion. The imaging portion is made from material that does not interfere with imaging transmissions, such as x-rays and MRI transmissions (e.g., made from x-ray compatible and/or MRI compatible materials). At least one foot alignment device is on the base. The foot alignment device includes at least one foot positioner with an inner wall and a toe wall, the inner wall extending medially away from the toe wall.

### RELATED APPLICATIONS

This application is related to and claims priority from U.S. Provisional Application No. 61/932,535, filed on Jan. 28, 2014, the disclosure of which is incorporated herein by reference in its entirety.

### FIELD OF THE INVENTION

The present invention is directed to imaging apparatus and, more particularly, to an alignment frame for use in a weight bearing imaging system for knees.

### BACKGROUND

Conventional three-dimension imaging of a part of the body, such as the knee, is generally accomplished by having the patient lie supine on a table while the scan is taken around the area of interest. The problem with such scanning techniques is that the area being scanned is, in many cases, not in a position where it is subjected to loads under which it normally operates. Thus, the scan is not a “real-life” image of the knee. Scanning an area, such as a joint, under load or in different positions permits capture of the relative positions of the various components of the joint (muscles, bones, cartilage, tendons, meniscus, etc.), thereby potentially highlighting issues that an unloaded view may not expose.

### SUMMARY OF THE INVENTION

The present invention is directed to a device for use in imaging, such as computer tomography imaging (CT) of a knee, upper calf or thigh region, while under load. The device includes an alignment frame configured to orient the leg in a prescribed position so as to permit the proper imaging of the knee, thigh or upper calf.

The alignment frame includes a base with a support extending upward from a base. A thigh positioner is attached to the support. The thigh positioner includes a panel portion for contact by a user's thigh, and an imaging portion located below the panel portion. The imaging portion is made from material that does not interfere with imaging transmissions, such as x-rays and MRI transmissions, for example x-ray compatible materials, such as wood or plastic, and/or MRI compatible materials, such as non-ferromagnetic materials. At least one foot alignment device is on the base. The foot alignment device includes at least one foot positioner with an inner wall and a toe wall, the inner wall extending medially away from the toe wall.

In one embodiment, the alignment frame includes a vertical support portion attached to the base and an adjustable horizontal support portion, the horizontal support portion permitting translation of the thigh positioner relative to the vertical support portion and the foot positioner.

The adjustable horizontal support portion may include a main section that is mounted to and extends laterally to the side of vertical support and a movable section is mounted to the main section such that the movable section can be translated relative to the main section. The thigh positioner is mounted to the distal end of the movable section thereby permitting adjustment of the horizontal spacing between the thigh positioner and the vertical support portion by sliding the movable section toward and away from the main section.

The movable section may include two spaced apart rails that slide along and engage with complementary rails formed on the main section.

The thigh positioner preferably includes a bracket mounted to a back side of the panel potion and is attached to the end of the movable section.

A locking mechanism may be mounted to the horizontal support portion, the locking mechanism configured to lock or inhibit movement of the movable section relative to the main section. The locking mechanism may be a ratcheting assembly that includes at least one rail with teeth mounted to the inside of the main section and a latch pivotally mounted on the movable section and configured to engage between the teeth A spring or gravity biases the latch into engagement with the teeth such that when engaged the movable section is inhibited from sliding toward the main section. An unlocking lever connected to the latch and mounted such that rotation of the lever causes the latch to lift against the bias of the spring or gravity and move out of engagement with the teeth.

In one embodiment, the vertical support portion includes a fixed lower frame and a movable upper frame. A lift motor is mounted to the fixed lower frame and engages with a lead screw that is connected to the upper frame and, as such, operation of the lift motor will cause the movable upper frame to raise and lower relative to the fixed lower frame.

The frame may include a hip bar assembly mounted to the moveable section and that includes two laterally spaced apart and adjustable hip bars mounted to an adjustment bar.

The foregoing and other features of the invention and advantages of the present invention will become more apparent in light of the following detailed description of the preferred embodiments, as illustrated in the accompanying figures. As will be realized, the invention is capable of modifications in various respects, all without departing from the invention. Accordingly, the drawings and the description are to be regarded as illustrative in nature, and not as restrictive

### BRIEF DESCRIPTION OF THE DRAWINGS

For the purpose of the illustrating the invention, the drawings show a form of the invention, which is presently preferred. However, it should be understood that this invention is not limited to the precise arrangements and instrumentalities shown in the drawings.

FIG. 1 is a top perspective view of one embodiment of an alignment frame according to the present invention.

FIG. 1A is a an exploded top perspective view of the embodiment of an alignment frame in FIG. 1.

FIG. 2 is a bottom perspective view of the alignment frame in FIG. 1.

FIG. 3 is an enlargement of a locking mechanism for mounting the vertical thigh positioner to the base.

FIG. 4 is a bottom view of the alignment frame of FIG. 1.

FIG. 5 is a cross-sectional view of the alignment frame of FIG. 1 taken along lines 5-5 in FIG. 4.

FIG. 6 is a schematic view of the alignment frame of FIG. 1 with a representation of a patient's leg standing on the frame during use.

FIG. 7 is a top perspective view of second embodiment of an alignment frame according to the present invention.

FIG. 8 is a perspective view of the alignment frame of FIG. 7 illustrating the lift motor.

FIG. 9 is a top view of the foot positioner, illustrating placement of a person's feet.

FIG. 10 is a front view of the alignment frame of FIG. 7.

FIG. 11 is a side view of the alignment frame of FIG. 7.

FIG. 12 is a bottom perspective view of the alignment frame of FIG. 7.

FIG. 12A is an enlarged view of the locking mechanism of FIG. 12.

FIG. 13 is a top view of the alignment frame of FIG. 7.

FIG. 13A is an enlarged view of the measurement mechanism of FIG. 13.

### DETAILED DESCRIPTION OF THE DRAWINGS

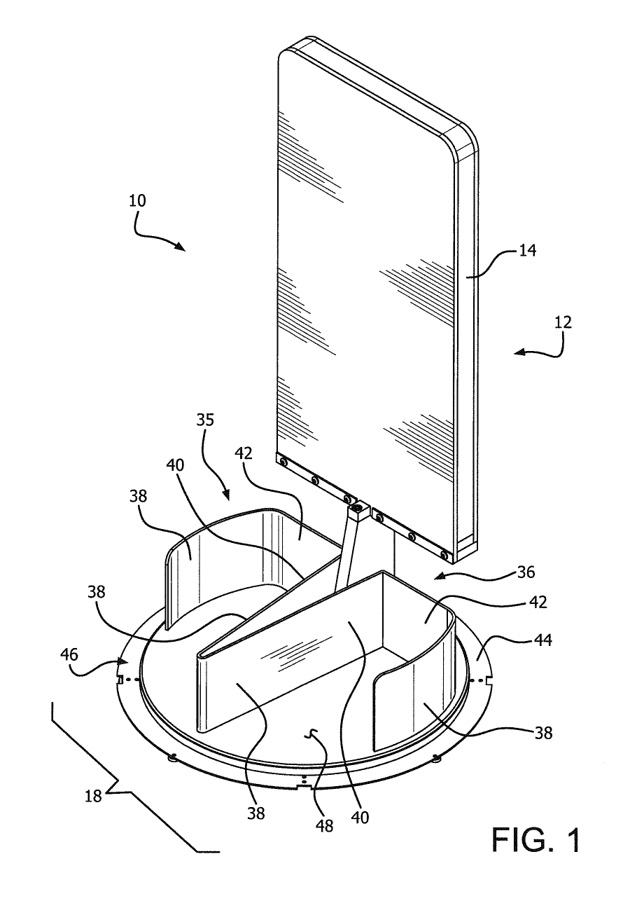

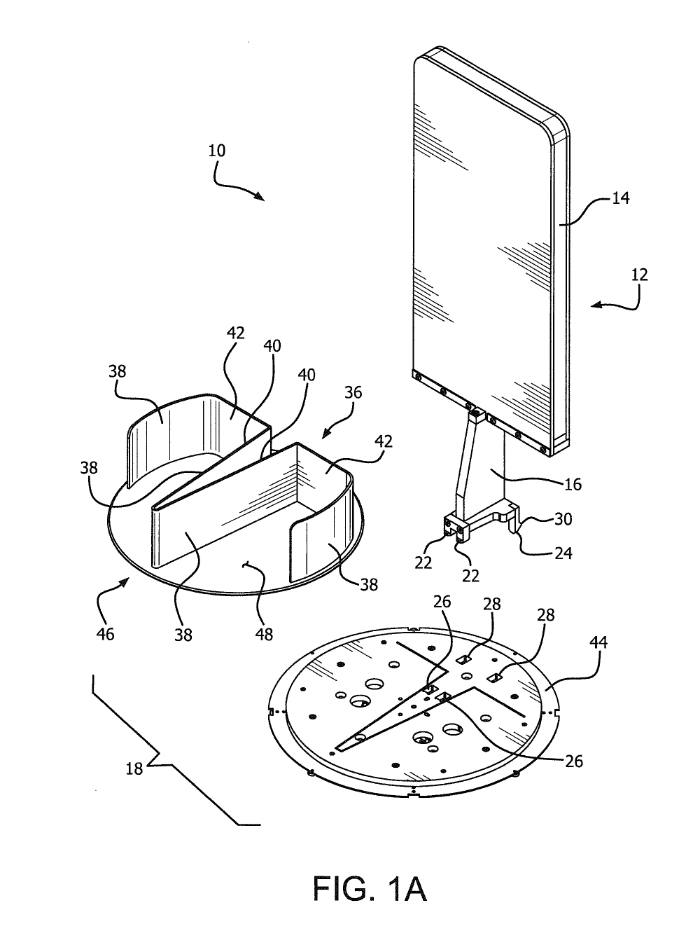

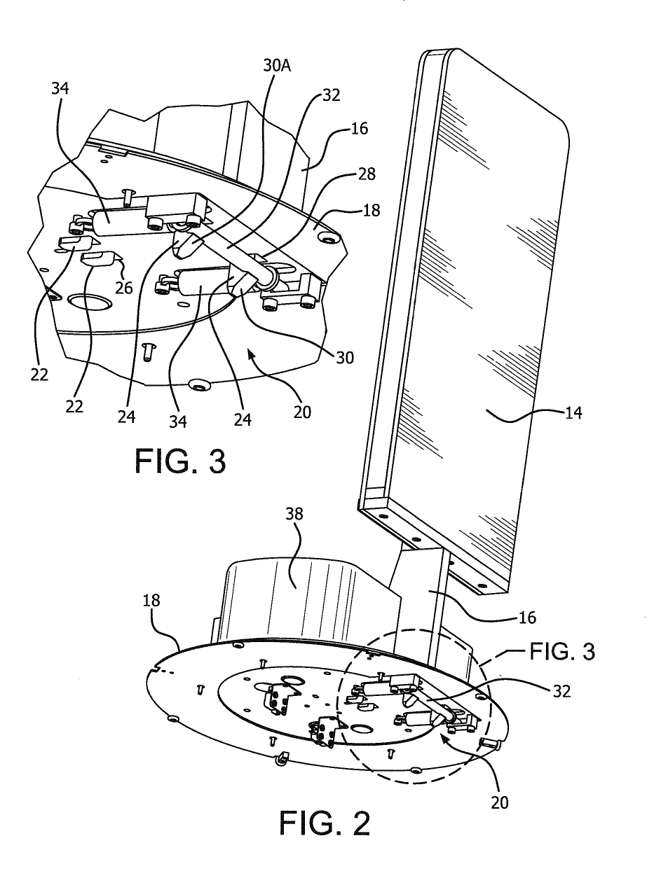

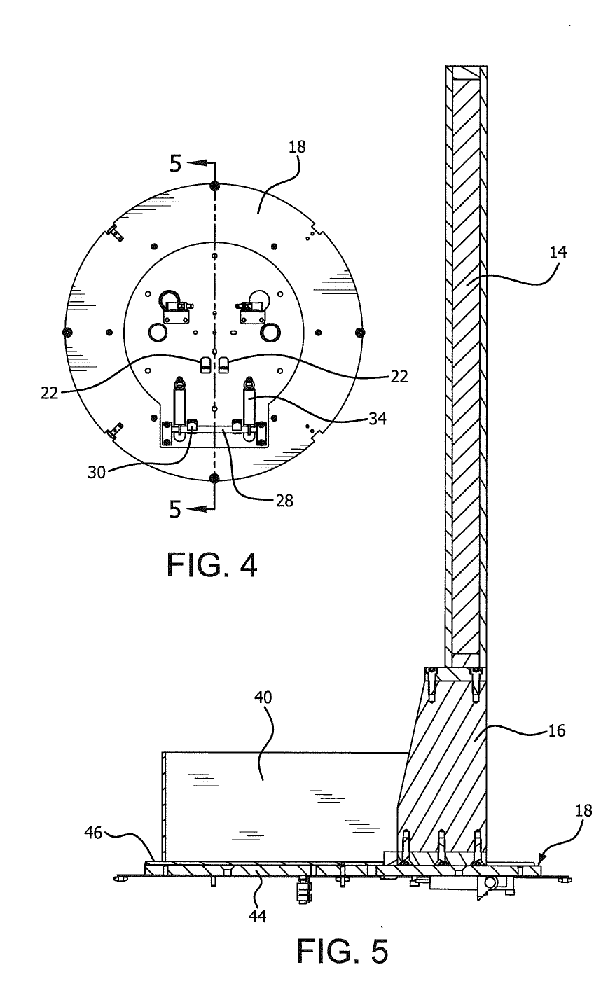

Referring now to FIGS. 1-6 of the drawings, where like reference numerals indicate similar components, an embodiment of an alignment frame 10 according to the invention is illustrated. The frame 10 includes a vertical thigh positioner 12, which is depicted in the figures as a panel portion 14, and a support 16. Although the panel portion 14 is shown as being planar, it should be readily understood that the panel portion 14 could have contours designed to conform to and orient the upper thigh of a patient for proper positioning as discussed below. The support 16 attaches the panel portion 16 to a base 18. At least a portion of the panel portion 14 is preferably made from a material that is material that does not interfere with imaging transmissions, such as x-ray and MRI transmissions, for example x-ray compatible materials, such as wood or plastic, and/or MRI compatible materials, such as non-ferromagnetic materials, and is also minimally flexible. Of course, it could be made from other materials such as carbon fiber. The portion that does not interfere with imaging transmissions is preferably located around the position of the patient's knee.

In one embodiment, the support 16 is removably attached to the base 18 such as through a locking mechanism 20 so that the thigh positioner 12 can be removed from the base for transport or storage. The locking mechanism 20 can be any conventional mechanism known to those skilled in the art for removably attaching a structure, such as nuts and bolts. However, in one preferred embodiment, which is shown more clearly in FIGS. 1A, 3 and 4, the locking mechanism 20 includes a one or more forward extensions 22 and one or more rear extensions 24 that each extend downward from the bottom of the support 16. The forward extensions 22 are sized to mate with corresponding openings 26 formed in the base 18. The rear extensions 24 are sized to mate with corresponding openings 28 formed in the base 18. As shown in FIGS. 1A and 3, the rear extensions 24 include rearwardly facing locking teeth 30. The teeth 30 are configured to engage with a locking bar 32 located on the bottom of the base 18. As should be apparent, when the front extensions 22 and the rear extensions 24 are in their respective openings 26, 28 and the teeth 30 are engaged with the locking bar 32, the thigh positioner 12 is attached to the base 18.

As shown in FIG. 1A, the base may include a lower support frame 44 and an upper portion 46. The upper portion 46 includes a top surface 48 and the foot positioners 36. The upper portion 46 may be attached to the support frame 44 through any conventional means.

One or more springs 34 preferably attach the locking bar 32 to the base 18. The teeth 30 preferably include a sloped engagement surface 30A. Accordingly, when the support 16 is being attached to the base, the sloping surface 30A of the teeth 30 contact the locking bar 32. The angle of the sloping surface 30A causes the locking bar 32 to move rearward, stretching the springs 34 until the teeth 30 clear the bar 32. The spring force from the stretched springs 34 pulls the locking bar 32 forward, capturing and locking the rear extensions in place. A disengagement latch (not shown) may connect to the locking bar 32 and extent to the top of the base so as to permit manual movement of the locking bar 32 for disengaging the thigh positioner 12 from the base 18.

At least one foot alignment device 35 is mounted to or formed integral with the top of the base 18. The foot alignment device preferably includes at least one and more particularly two foot positioners 36, one located on either side of the center of the base 18, which is preferably aligned with the center of the thigh positioner 12. In the illustrated embodiment, the two foot positioners are formed integral with or attached to one another. Each foot positioner 36 includes vertical walls or brackets 38, with an inner wall 40 and a toe wall 42. The inner wall 40 extends medially away from the toe wall 42 and toward the centerline of the thigh positioner preferably forming an angle of about 5 degrees. In the preferred embodiment where there are two adjacent foot positioners, the angle formed between the two adjacent inner walls 40 is approximately 10 degrees (see FIG. 9). The inner wall may be attached to the toe wall 42. The toe wall 42 is preferably located directly below the thigh positioner 12 so that they are vertically aligned. The toe wall 42 may be fixed in position on the base 18 or could be adjustable for different patients as will become more apparent below. The toe wall 42 preferably is about 4 inches high, although other heights are possible.

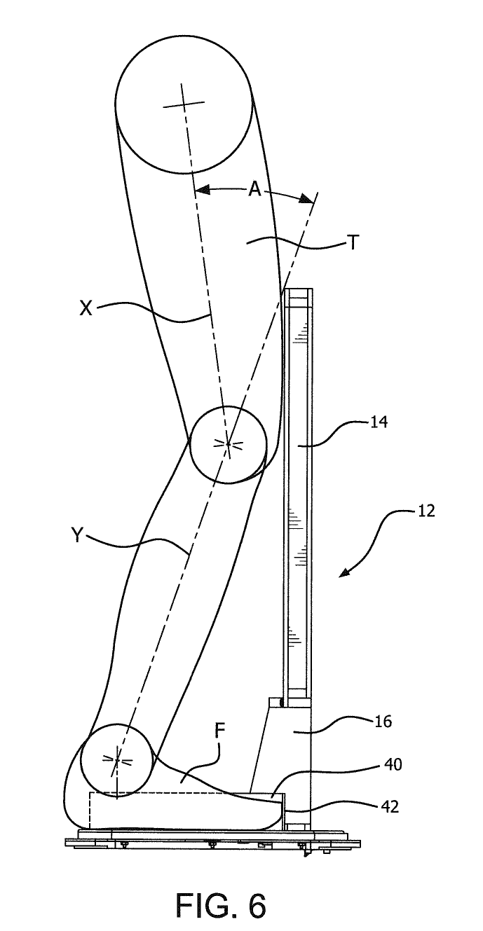

As shown in FIG. 6, the location of the toe wall 42 relative to the thigh positioner 12 in each foot positioner 36 is configured so that, when a patient's foot F is in the foot positioner 36 with their toes against the toe wall 42, and their forward thigh T (e.g., rectus femoris muscle) resting completely against the panel portion 14 of the thigh positioner 12, an axis X extending between the patient's hip and knee (e.g., approximate central axis of the upper leg) forms an angle A of about 20 degrees relative to an axis Y extending between the patient's knee and ankle (e.g., approximate central axis of the lower leg). This angle has been generally accepted in the industry as a standard position for weight-bearing knee radiography. Preferably the alignment frame 10 is adjustable so as to enable imaging of the knee at angle A between approximately 0 degrees and 30 degrees. This positioning results in a proper opening of the knee so that the tibiofemoral joint space can be imaged in the specific location in which the tibial or femoral cartilage most commonly is damaged in cases of osteoarthritis. Thus, the range of angles that the frame holds the knee are the critical angles for both accepted medical imaging of the knee as well as most sensitive for detection of early signs of knee osteoarthritis. Importantly, the features of the frame (foot positioner 36 with angled inner wall 40 and toe wall 42, in combination with the panel portion 14) enables reproducibility of these poses for weight-bearing knee imaging through fixing the foot, ankle, tibia bone, patella and thigh. Reliable reproduction of this pose enhances the ability to detect changes in the knee anatomy that are significant for diagnosis of development and progression (worsening) of knee joint pathology, including cartilage and meniscal loss as well as bony changes.

Although not shown in the first embodiment, it is contemplated that the device may include switches for controlling the vertical adjustment of the panel portion 14 relative to the base (for different height patients). Also, the lateral position of the toe wall 42 could be adjustable, or the horizontal position of the support 16 relative to the base could be adjustable to enable imaging at different knee flexion angle poses. The system may include straps for securing the thigh to the thigh positioner 12.

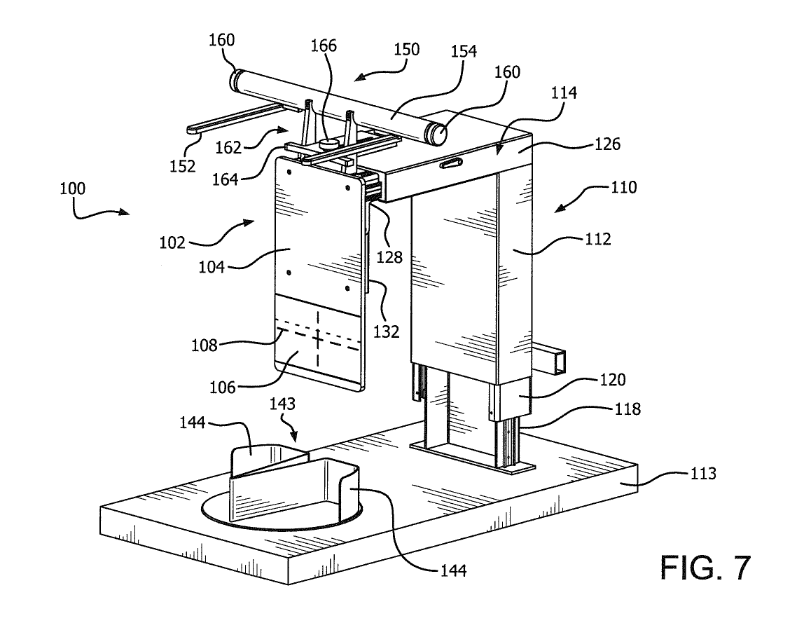

Referring now to FIG. 7, a second embodiment of the alignment frame 100 is shown. The alignment frame includes a thigh positioner 102 that preferably includes a upper thigh or panel portion 104 and a lower knee imaging portion 106. In this embodiment, only the knee imaging portion 106 (delineated in the figure by the solid horizontal lines) needs to be made from material that does not interfere with imaging transmissions as discussed above. The knee imaging portion 106 is preferably attached to the panel portion 104. The knee imaging portion may include markings 108 which assist medical staff in aligning portions of a patient's body. As with the first embodiment, the panel portion may include contours to facilitate positioning of the patient's thigh.

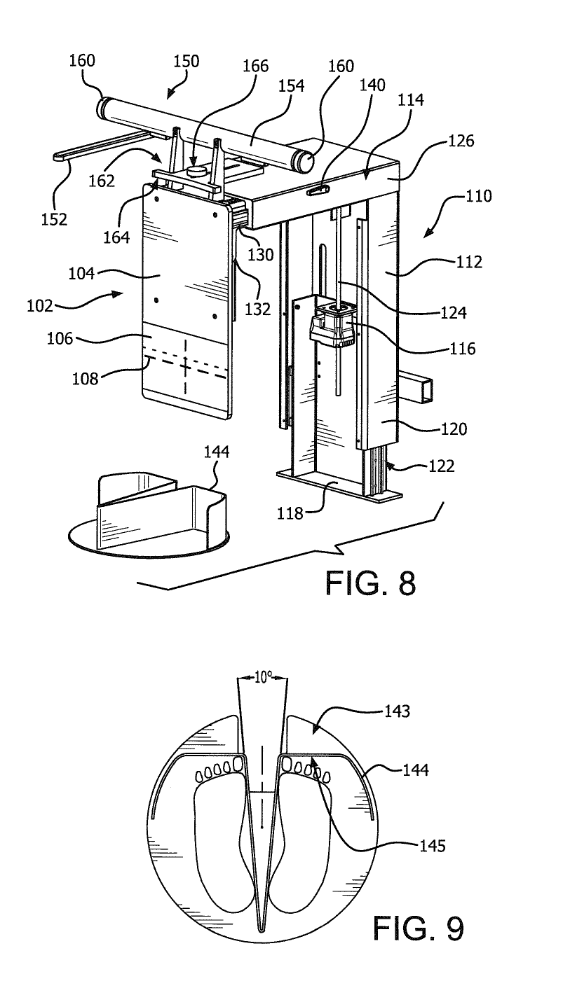

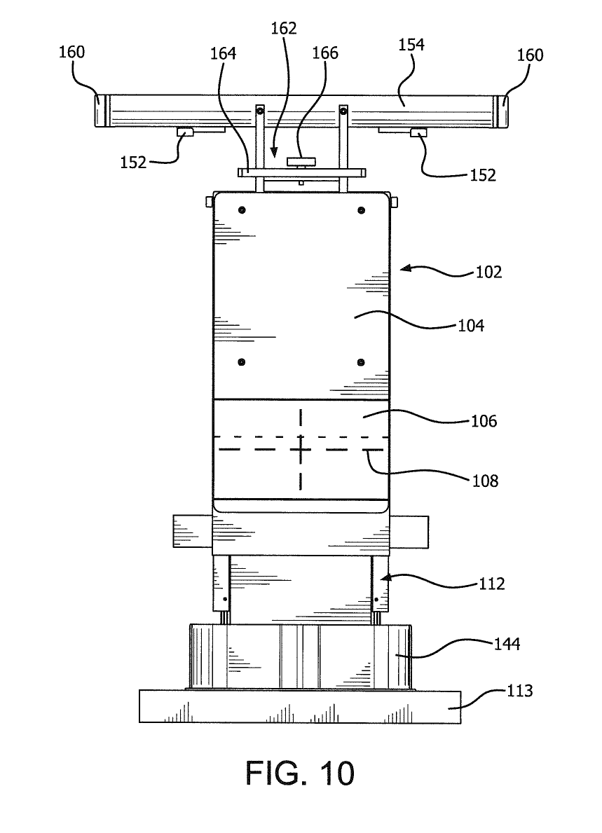

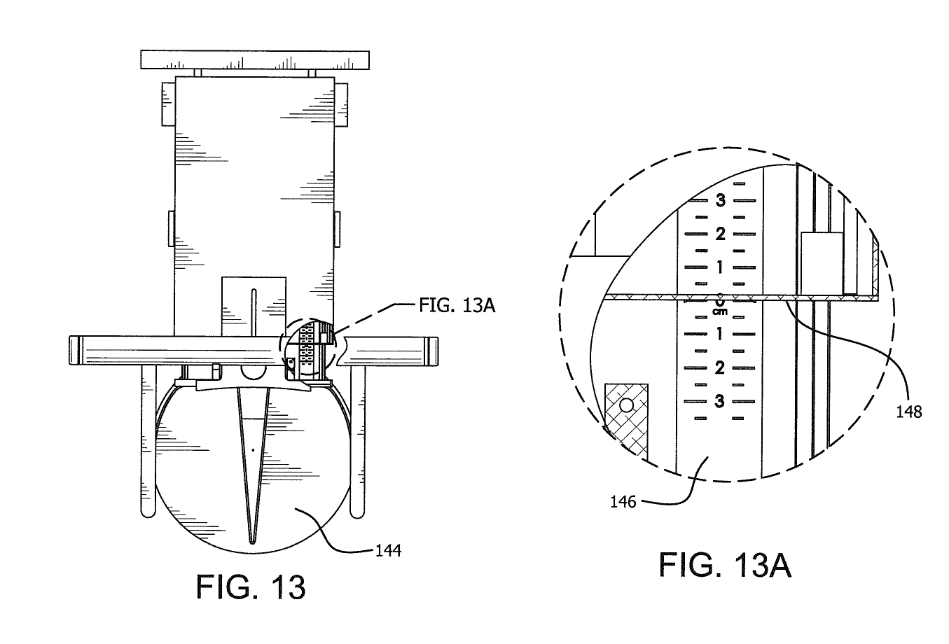

The thigh positioner 102 is attached to a frame support 110. In one configuration, the frame support 110 includes an adjustable vertical support portion 112 and an adjustable horizontal support portion 114. The vertical support portion 112 preferably attaches to a base 113 (not shown in FIGS. 8, 9, 13 and 13.) To provide vertical adjustment, in one configuration, a lift motor 116 raises and lowers the horizontal support portion 114. In the illustrated embodiment, the vertical support portion includes a fixed lower frame 118 and a movable upper frame 120. Guide rails 122 may be mounted to vertical arms of the fixed lower frame 118 which engage with mating rail members on vertical arms of the upper frame 120. The lift motor 116 is preferably mounted to the fixed lower frame 118 and engages with a lead screw 124 that is connected to the upper frame 120. As should be apparent, operation of the lift motor will cause the movable upper frame 120 to raise and lower relative to the fixed lower frame 118. Preferably the system permits about 22 cm of height adjustment. Although a lift motor and screw drive system are illustrated, it should be readily apparent that other lifting mechanisms can be used, such as a mechanical drive, pneumatic lifts, ratchet drive, etc.

The adjustable horizontal support portion 114 preferably includes a main section 126 that is mounted to and extends laterally to the side of the upper frame 120 such that the main section 126 is raised and lowered with the upper frame 120. A movable section 128 is mounted to the main section 126 such that the movable section 128 can be translated relative to the main section 126. In the illustrated embodiment, the movable section includes two spaced apart rails 130 that slide along complementary rails formed on the main section 126.

The thigh positioner 102 is mounted to the distal end of the movable section 128. As shown, a bracket 132 is mounted to the back of the thigh positioner 102 and attached to the end of the movable section 128. As such, the horizontal spacing between the thigh positioner 102 and the vertical support portion 112 is changed by sliding the movable section 128 toward and away from the main section 126.

Since during use a patient is required to rest their thigh against the panel portion 104 of the thigh positioner 102, it is desirable to have a locking mechanism 134 to lock or inhibit movement of the movable section 128 relative to the main section 126. In the illustrated embodiment, the locking mechanism 134 is a ratcheting assembly that includes at least one rail with teeth 136 mounted to the inside of the main section 126 and a latch 138 pivotally mounted on the movable section 128 and configured to engage between the teeth 136. A spring (not shown) may be used to bias the latch 138 into engagement with the teeth 136. Alternatively, the latch can be designed such that gravity provides a sufficient biasing force for engagement. Once engaged, the movable section 128 is inhibited from sliding toward the main section. In the illustrated embodiment, the ratcheting assembly 134 prevents movement of the movable section in only one direction (toward the main section when the latch is engaged with the teeth). The movable section can be slid away from the main section causing the latch to ride along the back of the teeth sliding out of engagement and then snapping into engagement with the next teeth.

In order to permit the movable section 128 to move inwards toward the main section 126, the ratcheting assembly 134 includes a unlocking lever 140 that is connected to the latch 138. Rotating the lever causes the latch to lift against the bias of the spring or gravity and move it out of engagement with the teeth. While the unlocking lever 140 is held up, the movable section 128 can be slid in and out of the main section 126. Preferably there are two rails with teeth 136 on opposite sides of the main section and two latches, one engaging each rail. A connecting rod 142 connects the latches 138 to the unlocking lever 140. Of course it should be understood that any suitable system for translating the movable section relative to the main section can used in the present invention, include electric, pneumatic and other power driven systems.

As with the first embodiment, at least one foot alignment device 143 is mounted to or formed integral with the top of the base 18. The foot alignment device preferably includes at least one foot positioner 144, and more particularly two foot positioners 144, each foot positioner 144 located on one side of the centerline of the thigh positioner 102. Similar to that which is shown in FIG. 6, the location of the foot positioner 144 relative to the thigh positioner 102 is configured such that, when a patient's foot F is in the foot positioner 144 with their toes against the toe wall 145, and their forward thigh T (e.g., rectus femoris muscle) resting completely against the panel portion 104 of the thigh positioner 102, an axis X extending between the patient's hip and knee (e.g., approximate central axis of the upper leg) forms an angle A of about 20 degrees relative to an axis Y extending between the patient's knee and ankle (e.g., approximate central axis of the lower leg). This angle can be changed to vary the loading on the knee by sliding the movable section 128 inward and outward from the main section 126. Thus, angles between approximately 0 degrees (standing straight up) and 30 degrees or more may be obtained.

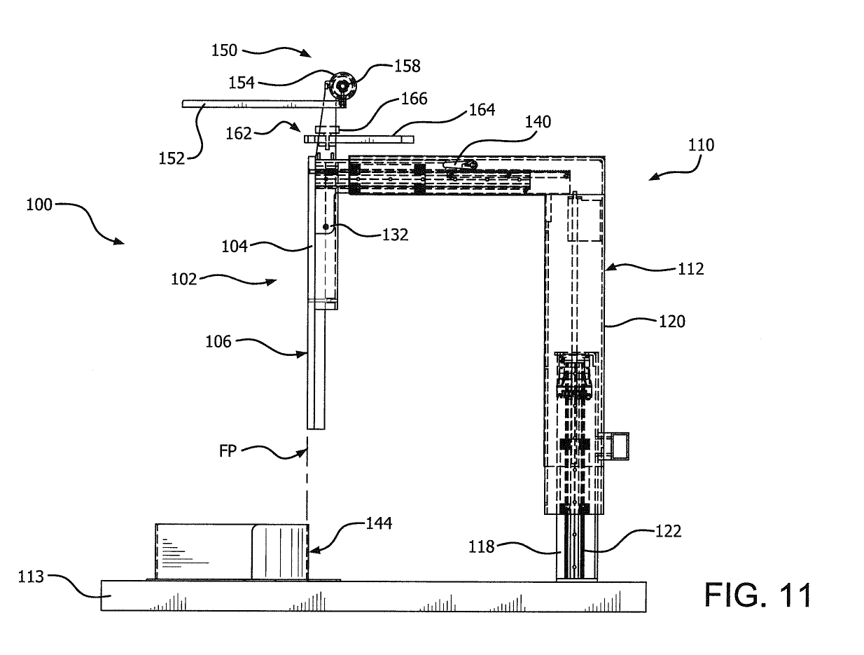

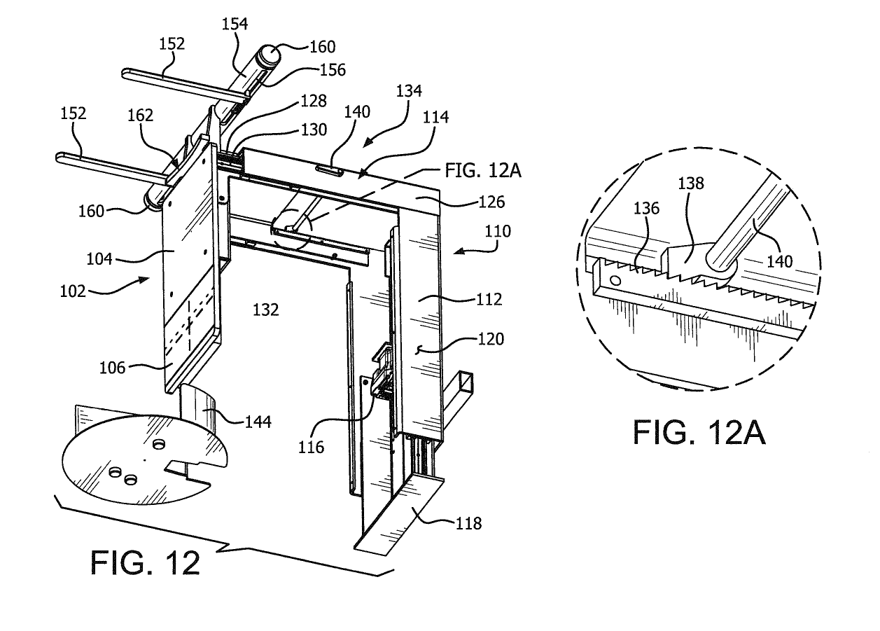

In order to accurately permit reproducibility of different knee poses for a patient, a measurement mechanism is preferably included. As such, a scale 146 is mounted to the movable section 128 where it slides past a vertical wall 148 on the main section 126. The teeth 136 on the rail are preferably set so that each engagement by the latch 138 corresponds to 5 mm of movement. The zero position of the scale 146 is preferably set such that front plane FP of the knee plate 108 is aligned with the front plane of the toe plate as shown in FIG. 11. The movable section 128 can preferably move approximately 20.5 cm relative to the main section 126.

In one embodiment, the alignment frame 100 includes a hip bar assembly 150 that includes two laterally spaced apart and adjustable hip bars 152 that are intended to be placed against the sides of the hips of a patient. The hip bars 152 are mounted to an adjustment bar 154. The adjustment bar 154 is attached to movable section 128, more preferably to the bracket 132 on the movable section 128. A slot 156 extends along a portion of the bottom of the adjustment bar. A threaded adjustment rod 158 (shown in phantom in FIG. 11) is mounted inside the adjustment bar. The hip bars 152 are attached to the threaded adjustment rod 158. At least one and preferably two knobs 160 are attached to the threaded rod 158 such that rotation of the knobs 160 cause the hip bars 152 to move toward and away from one another.

A waist positioner 162 may be included to further assist with repeatability of body positioning for scans. The waist positioner 162 includes a waist bar 164 that is mounted to the movable section so that it can slide toward and away from the adjustment bar 154. In the illustrated embodiment, a threaded knob 166 is mounted in a slot in the waist bar 164. By tightening the knob 166, the waist bar 164 can be locked into a position.

One of the benefits of the present invention is that it permits reliable repositioning of the patient on serial examinations. This results in more accurate diagnostic analysis of a patient's condition. Also, the ability of the patient to lean against the thigh positioner enhances the patient's comfort during the imaging by providing better balance, thus minimizing patient motion artifacts.

For the purposes of promoting an understanding of the principles of the invention, reference has been made to the preferred embodiments illustrated in the drawings, and specific language has been used to describe these embodiments. However, no limitation of the scope of the invention is intended by this specific language, and the invention should be construed to encompass all embodiments that would normally occur to one of ordinary skill in the art.

The particular implementations shown and described herein are illustrative examples of the invention and are not intended to otherwise limit the scope of the invention in any way. For the sake of brevity, conventional electronics, control systems, software development and other functional aspects of the systems (and components of the individual operating components of the systems) may not be described in detail.

Finally, the use of any and all examples, or exemplary language (e.g., “such as”) provided herein, is intended merely to better illuminate the invention and does not pose a limitation on the scope of the invention unless otherwise claimed. Numerous modifications and adaptations will be readily apparent to those skilled in this art without departing from the spirit and scope of the invention.

1. An alignment frame for use in weight bearing imaging of a knee, the alignment frame comprising:
a base;
a support extending upward from a base;
a thigh positioner attached to the support, the thigh positioner including a panel portion for contact by a user's thigh, and an imaging portion located below the panel portion, the imaging portion being made from material that does not interfere with imaging transmissions; and
at least one foot alignment device on the base, the foot alignment device including at least one foot positioner, the foot positioner includes a vertical wall with an inner wall and a toe wall, the inner wall extending medially away from the toe wall.

2. The alignment frame according to claim 1, wherein the foot alignment device includes two foot positioners, one located on either side of a center of the thigh positioner.

3. The alignment frame according to claim 2, the two foot positioners are formed integral with another.

4. The alignment frame according to claim 1, wherein the support is removably attached to the base.

5. The alignment frame according to claim 1, wherein support includes a vertical support portion attached to the base and an adjustable horizontal support portion, the horizontal support portion permitting translation of the thigh positioner relative to the vertical support portion and the foot positioner.

6. The alignment frame according to claim 5, wherein the adjustable horizontal support portion includes a main section that is mounted to and extends laterally to the side of vertical support and a movable section is mounted to the main section such that the movable section can be translated relative to the main section, the thigh positioner is mounted to the distal end of the movable section thereby permitting adjustment of the horizontal spacing between the thigh positioner and the vertical support portion by sliding the movable section toward and away from the main section.

7. The alignment frame according to claim 6, wherein the movable section includes two spaced apart rails that slide along and engage with complementary rails formed on the main section.

8. The alignment frame according to claim 5, wherein the thigh positioner includes a bracket mounted to a back side of the panel potion and is attached to the end of the movable section.

9. The alignment frame according to claim 5, further comprising a locking mechanism mounted to the horizontal support portion, the locking mechanism configured to lock or inhibit movement of the movable section relative to the main section.

10. The alignment frame according to claim 9, wherein the locking mechanism is a ratcheting assembly that includes at least one rail with teeth mounted to the inside of the main section and a latch pivotally mounted on the movable section and configured to engage between the teeth, the latch being biased into engagement with the teeth such that when engaged the movable section is inhibited from sliding toward the main section, and an unlocking lever connected to the latch and mounted such that rotation of the lever causes the latch to lift out of engagement with the teeth.

11. The alignment frame according to claim 5, wherein vertical support portion includes a fixed lower frame and a movable upper frame, a lift motor is mounted to the fixed lower frame and engages with a lead screw that is connected to the upper frame and, as such, operation of the lift motor will cause the movable upper frame to raise and lower relative to the fixed lower frame.

12. The alignment frame according to claim 5, further comprising a hip bar assembly with two laterally spaced apart and adjustable hip bars mounted to an adjustment bar.

13. An alignment frame for use in weight bearing imaging of a knee, the alignment frame comprising:
a base;
a support extending upward from a base;
a thigh positioner attached to the support, the thigh positioner including a panel portion for contact by a user's thigh, the panel portion lying along a first plane, and an imaging portion located below the panel portion, the imaging portion being made from material that does not interfere with imaging transmissions; and
at least one foot alignment device on the base, the foot alignment device including at least one foot positioner, the foot positioner including a vertical wall with an inner wall and a toe wall, the inner wall extending medially away from the toe wall, the foot alignment device being positioned relative to the support such that the toe wall lies along a plane that is aligned with a first plane.

14. The alignment frame according to claim 1, wherein the relative position between the toe wall of the foot alignment device and the first plane is adjustable such that in a second position the toe wall lies along a plane that is not aligned with a first plane.

15. The alignment frame according to claim 13, wherein the support is removably attached to the base.

16. The alignment frame according to claim 13, wherein support includes a vertical support portion attached to the base and an adjustable horizontal support portion, the adjustable horizontal support portion permitting translation of the thigh positioner relative to the vertical support portion and the foot positioner, the adjustable horizontal support portion including a main section that is mounted to and extends laterally to the side of vertical support, and a movable section mounted to the main section such that the movable section can be translated relative to the main section, wherein the thigh positioner is mounted to the distal end of the movable section thereby permitting adjustment of the horizontal spacing between the thigh positioner and the vertical support portion by sliding the movable section toward and away from the main section.

17. The alignment frame according to claim 16, further comprising a locking mechanism mounted to the horizontal support portion, the locking mechanism configured to lock or inhibit movement of the movable section relative to the main section, the locking mechanism comprising a ratcheting assembly that includes at least one rail with teeth mounted to the inside of the main section and a latch pivotally mounted on the movable section and configured to engage between the teeth such that when engaged the movable section is inhibited from sliding toward the main section, and an unlocking lever connected to the latch and mounted such that rotation of the lever causes the latch to lift out of engagement with the teeth.

18. The alignment frame according to claim 17, wherein vertical support portion includes a fixed lower frame and a movable upper frame, a lift motor is mounted to the fixed lower frame and engages with a lead screw that is connected to the upper frame and, as such, operation of the lift motor will cause the movable upper frame to raise and lower relative to the fixed lower frame.

19. The alignment frame according to claim 18, further comprising a hip bar assembly with two laterally spaced apart and adjustable hip bars mounted to an adjustment bar.
